(19) United States
(12) Patent Application Publication
(10) Pub. No.: US 2011/0288113 A1
(43) Pub. Date: Nov. 24, 2011

(54) METHOD TO REDUCE OR ELMINATE THE USE BY A PATIENT OF A TOLERANCE-INDUCING PHARAMACOLOGICAL AGENT

(76) Inventor: Stephen J. Peroutka, San Jose, CA (US)

(21) Appl. No.: 12/952,823

(22) Filed: Nov. 23, 2010

Related U.S. Application Data
(60) Provisional application No. 61/265,360, filed on Dec. 1, 2009.

(51) Int. Cl. A61K 31/485; A61P 25/04; A61K 31/135; A61K 31/137; A61K 31/4468; A61K 31/451

(52) U.S. Cl. 514/282; 514/329; 514/289; 514/330; 514/648; 514/654; 514/646

(57) ABSTRACT

A multiple total daily dose regimen is developed in order to decrease the use by a patient of a tolerance-inducing pharmacological agent. In one example, the method reduces or phases out altogether a patient's chronic use of opioid drugs by use of a successive reduction of the total daily dose of an opioid drug in sub-therapeutic decrements over the course of 3-7 days per therapeutic dose.

This application claims the benefit of U.S. Application No. 61/265,360, filed Dec. 1, 2009. This application is incorporated by referenced herein in its entirety.

The present invention is directed to a method of reducing or eliminating the use by a patient of a tolerance-inducing pharmacological agent. Specifically, in one example, the invention is a method to reduce or phase out a patient's chronic use of opioid drugs by use of a successive reduction of the opioid drug dose in sub-therapeutic decrements.

### BACKGROUND

Opioid drugs comprise the major class of analgesic agents. They have provided significant analgesic benefits to millions of people over thousands of years. Their clinical utility in multiple painful clinical conditions is not debatable. However, the extended use of opioid drugs is associated with both the development of tolerance (requiring a progressive increase in dose to maintain analgesic efficacy) and/or a physiological dependence on the drugs (as a result of molecular changes in opioid receptor pathways). The net clinical result is not just a probable diminished analgesic effect over time but also a physiological dependence on opioid drugs that is distinct from their initial primary analgesic effects.

The clinical use of opioids for an extended period of time is also associated with a number of risks and untoward side effects. Physiological opioid dependence (often referred to as “addiction”) can be defined as the absolute need for continued drug use in order to maintain normal physiological function. It is a complex condition with pharmacologic, genetic and psychosocial elements. Psychological “addiction” (also described as drug craving and/or psychic dependence) is another type of opioid dependence that is usually differentiated from physiological dependence. However, all of the types of “dependence” imply an inability to decrease or discontinue opioid use. Withdrawal of opioids in a dependent individual may result in a distinct physiological condition that may cause considerable discomfort.

A functional tolerance to the pharmacodynamic effects of opioids is commonly observed in humans. Tolerance can be defined as a reduction in sensitivity to a given amount of a drug after repeated use. Opioid tolerance is a pharmacologic phenomenon that can result from the repeated use of opioids over an extended period of time (i.e., generally weeks or longer). Dose escalation with chronic opioid use is common. Indeed, tolerance results in the need to increase the opioid doses every few months, on average, to maintain equipotent analgesic effects.

There is another major clinical reason to discontinue chronic opioid use that is rarely stated in medical guidelines: the underlying painful disease process may have improved. As a result of surgery, exercise, natural progression and/or other interventions, opioids may no longer be required for their analgesic effects.

A variety of opioid withdrawal methods (often called detoxification or “detox”) have been proposed. Abrupt cessation of chronic high dose opioid use can produce an intense withdrawal syndrome in humans. More commonly, withdrawal of opioids utilizes a tapering protocol. There is no single protocol that has been accepted as more efficacious than another and the limited number of suggested withdrawal methods that have been described have some important practical limitations. For example, one practice guideline on chronic opioid therapy suggests tapering regimens for several different opioids that use available opioid formulations and that involve intermittent dose decreases in therapeutic dose steps while maintaining the same dose for multiple days.

### SUMMARY

Accordingly, it is an object of the present invention to provide an efficient and effective method that will decrease the use by a patient of a tolerance-inducing pharmacological agent. The method may reduce or phase out altogether a patient's chronic use of opioid drugs by use of a successive reduction of the total daily dose, defined as the total amount of an opioid drug taken per day, of an opioid drug in sub-therapeutic dose decrements, defined as an amount of opioid drug that is less than the amount of a therapeutic dose of the same opioid drug.

In one example, a method for decreasing the use of tolerance-inducing pharmacological agents comprises the steps of identifying a tolerance-inducing pharmacological agent and identifying a therapeutic dose amount of the tolerance-inducing pharmacological agent, the therapeutic dose amount being the minimum amount of the tolerance-inducing pharmacological agent required to accomplish a therapeutic purpose. The method further includes creating a multiple dose regimen of the tolerance-inducing pharmacological agent that comprises a plurality of successively smaller total daily dose amounts of the tolerance-inducing pharmacological agent, with the total daily dose amounts decreasing at a substantially steady rate. Further the total daily dose amount decreases are in decrements of sub-therapeutic amounts of the tolerance-inducing pharmacological agent. The tolerance-inducing pharmacological agent may be selected from the group consisting of opioids and benzodiazepines. The dosing regimen may comprise a single or multiple doses per day of the tolerance-inducing pharmacological agent, and may further comprise a decreasing total daily dose amount for each day of the regimen. The total daily dose of the tolerance-inducing pharmacological agent may be reduced to zero at the end of the regimen. The successively smaller sub-therapeutic dose amount decrements may be less than or equal to about 33.3% of the therapeutic dose. The total daily dose may be in oral formulation selected from the group consisting of a pill, capsule, tablet, lozenge, aerosol or trans-oral formulation. The dosing taper regimen for a single therapeutic dose amount may last from 3 to 7 days, with the total length of the taper process dependent upon the initial total daily dose amount.

### BRIEF DESCRIPTION OF THE DRAWINGS

FIG. 1 is a graphical comparison of prior art therapeutic does step tapering compared with an example sub-therapeutic dose tapering method.

### DETAILED DESCRIPTION

At the present time, there have been no known systematic attempts to decrease tolerance-inducing drug usage using a standardized tapering approach. Indeed, there is no standard protocol for tapering opioids, the most commonly used tolerance-inducing prescription drugs. Moreover, the few suggested guidelines that do exist are limited by available dosage formulations of pills, capsules and transdermal patches, for example, that do not allow for a gradual, slow and steady decrease in opioid dosage.

The present invention includes a sub-therapeutic dose tapering method for tolerance-inducing drugs that is based on the following observations:
Tolerance to therapeutic doses of drugs (such as opioids and benzodiazepines) can develop gradually over a period of days to weeks with chronic human use.
Withdrawal symptoms usually begin within hours after discontinuation of chronic treatment with tolerance-inducing drugs, peak within 2-3 days, and then gradually dissipate over the next few days.
Molecular changes resulting from a wide variety of chronic drug treatments (e.g. opioids and benzodiazepines) occur over a period of days to weeks.
   Therefore, the following sub-therapeutic dose tapering method was developed to provide a gradual decrease in dose that mirrors the time course of the gradual clinical and molecular changes that occur with the chronic use of tolerance-inducing pharmacological agents while avoiding a decrease in dose that induces withdrawal symptoms.

Although the discussion below is focused primarily on opioids, this is exemplary and the methods can be applied to other tolerance inducing pharmacological agents. For example, benzodiazepines are another class of tolerance-inducing pharmacological agents.

Since opioid-induced molecular and physiological changes related to withdrawal have been observed over a period of days after opioid discontinuation, the examples of a sub-therapeutic taper method avoids an acute decrease in total daily opioid dose that represents a minimum therapeutic dose amount or greater. More specifically, this method is designed to taper a single therapeutic dose of an opioid over a period of days (i.e. 3-7 days) in order to avoid and/or significantly reduce the abrupt molecular and physiological changes that can result from a more rapid (i.e., an entire therapeutic dose amount or greater) decrease in total daily opioid dose.

The therapeutic dose of various opioid formulations used in the medical treatment of moderate to severe pain can be defined based on the minimal recommended single dose of various opioid formulations approved by the United States Food and Drug Administration for the treatment of moderate to severe pain. Examples are provided in Table 1 below of the minimally effective analgesic oral therapeutic doses for the treatment of moderate to severe pain that have been approved for 15 different formulations of 10 different opioids. The following table is exemplary of oral doses. Similar information and determination of minimum amounts of opioid (or other tolerance-inducing pharmacological agents) can be made with respect to intravenous, intramuscular and transdermal delivery methods.

TABLE 1

| | | Minimum Amounts of |
|---|---|---|
| | | the Opioid Required |
| Oral opioids approved for the | FDA approved total daily | to Accomplish a |
| treatment of moderate to severe | dosing regimens (from | Therapeutic Purpose |
| pain | Product Labels) | (mg) |
| hydrocodone | 5-10 mg q 4-6 h (60 mg/day max) | 5 |
| hydromorphone | 2-4 mg q 4-6 h | 2 |
| levorphanol | 2 mg q 6-8 h | 2 |
| meperidine | 50-150 mg q 3-4 h | 50 |
| methadone | 2.5-10 mg q 8-12 h | 2.5 |
| morphine (immediate release) | 15-30 mg q 4 h | 15 |
| morphine (extended release q12 h | 15-60 mg q 8-12 h | 15 |
| dosing) | | |
| morphine (extended release q24 h | 30 mg q d | 30 |
| dosing) | | |
| oxycodone (immediate release) | 5 mg q 6 h | 5 |
| oxycodone (immediate release) | 10 mg q 12 h | 10 |
| oxymorphone | 10-20 mg q 4-6 h | 10 |
| oxymorphone (extended release q12 | 5 mg q 12 h | 5 |
| h dosing) | | |
| tapendatol | 50-100 mg q 4-6 | 50 |
| tramadol (immediate release) | 50-100 mg q 4-6 | 50 |
| tramadol (extended release q24 h | 100 mg qd | 100 |
| dosing) | | |

The maximal allowable total daily dose rate of decrease can be calculated by dividing the therapeutic dose of a given drug formulation by the minimal number of days required to decrease a single therapeutic dose amount as described herein (i.e., 3 days). Therefore, by definition, the maximal total daily dose decrease allowed must be a sub-therapeutic dose amount of the drug (i.e., a dose less than a therapeutic dose). Based on the formula below, the maximal allowable single total daily dose decrease for this example must be no more than 33.3% of a therapeutic dose.

Maximal total daily dose decrease=Minimum Therapeutic dose/3

The total daily dose need not decrease daily if a single therapeutic taper takes place over a time period longer than 3 days but, for this example, all total daily dose decreases must be in dose decrements that are no more than 33.3% of a therapeutic dose.

Continuing with the above example of 33.3% therapeutic dose decrements, in the case of immediate release morphine, the maximal allowable total daily dose decrease for a 15 mg morphine therapeutic dose taper would be 5 mg/day over the course of 3 days. Slower rates of sub-therapeutic dose decrease are also acceptable. For example, a 15 mg morphine therapeutic dose can be decreased by 3 mg/day over the course of 5 days. As another example, a 15 mg morphine therapeutic dose can be decreased by 2.14 mg/day over the course of 7 days.

There does not exist currently a range of oral capsule or pill opioid dose formulations that would allow this sub-therapeutic dose taper to be reduced to practice with capsules or pills. For example, there are no marketed oral dose formulations of 5 mg morphine immediate release capsules or pills or of oxycodone capsule or pill doses below its therapeutic dose of 5 mg, levorphanol capsule or pill doses below its therapeutic dose of 2 mg, etc. Therefore, it is necessary to obtain a set of opioid dose formulations that allow this sub-therapeutic taper method to be reduced to practice.

Since the method described above is based on the rate of taper of a single minimum amount of opioid required to accomplish a therapeutic purpose (aka therapeutic dose), the total duration of the taper method is dependent upon the total daily dose of opioid actually used at the start of the taper process. The total daily dose of opioid used to treat moderate to severe pain varies between patients. The total daily dose of opioid used is also dependent upon a number of other factors such as the specific opioid used, its formulation and the duration of its use by the patient. The total duration (in days) required to taper an individual completely from any given total daily dose of an opioid can be calculated as follows: divide the total daily dose (in mg) taken at the onset of the taper by the therapeutic dose (in mg) (minimum amount required to accomplish a therapeutic purpose) of the drug and then multiple by 3-7 days. Thus, for an individual taking 60 mg per day of immediate release morphine, a total of 4 therapeutic doses of opioid (i.e. 4×15 mg therapeutic dose=60 mg immediate release morphine) should be tapered no faster than over a 12 (i.e., 4 therapeutic doses×3 days minimal taper period) to 28 (i.e., 4 therapeutic doses×7 days) taper period. For an individual taking 180 mg of immediate release morphine, a total of 12 therapeutic doses of opioid (i.e. 12×15 mg therapeutic dose=180 mg immediate release morphine) should be tapered no faster than over a 36 (i.e., 12 therapeutic doses×3 days minimal taper period) to 84 (i.e., 12 therapeutic doses×7 days) taper period. A slower rate of taper is also possible although not necessary to meet the goals of this taper regimen.

Sometimes a tolerance-inducing pharmacological agent is administered more than once a day (i.e. multiple therapeutic doses are taken at various time points throughout a day). Each total daily dose decrement, including each decremental dose that is given during a day, may be reduced by a sub-therapeutic amount as demonstrated. Alternatively, the sum of the dose amounts for each day may be less than the sum of the dose amounts of the immediately previous day. In some situations, the decremental decrease in the dose regimen may be the same for one or two consecutive days before there is a decrease in the sub-therapeutic dose amount. Accordingly, depending on the starting clinical dose of the pharmacological agent, the length of the treatment taper regimen may be three to seven days per therapeutic dose. Thus an individual patient taking 60 mg morphine as a clinical dose (i.e., a daily total of 4 therapeutic doses) would require a series of 4 sequential therapeutic dose tapers (i.e., 12-28 days) to reduce opioid use to zero.

The present method is described below, using specific examples of the method applied to various opioid drugs taken at an initial daily dose that represents 4 therapeutic doses (e.g., 60 mg immediate release morphine).

### 1. Determine if the Subject Patient is a Candidate for Opioid Dose Reduction

This method can be applied to any individual patient taking chronic opioids for whatever reason who wishes to decrease and/or discontinue the use of opioid medications. The method is also applicable to individuals who have reported stable and acceptable pain scores (e.g., ≦4 on a 0-11 Likert Pain Scale) for at least 3-6 months on the same dose of opioid.

### 2. Initiate Sub-Therapeutic Dose Taper

More generally, the tapering regime can be adjusted to any total daily dose that a patient is receiving and the total daily dose tapered over any desired time period. The following examples illustrate the tapering doses administered sequentially over a sample tapering period. The total daily dose can be reduced completely to zero, or to retain a therapeutic effect, to a much lower total daily dose than administered to the subject before.

TABLE 2

| | q 24 | | | | | | | | |
|---|---|---|---|---|---|---|---|---|---|
| | hour | q12 hour | | | | | | | |
| | mor- | morphine | | q12 hour | | | | q12 hour | |
| | phine | (e.g. MS | | hydro- | | q12 hour | | oxymorphone | |
| | (e.g., | Contin) | | codone | | oxycodone | | (e.g. Opana) | |
| | Avinza) | Am | PM | Am | PM | Am | PM | Am | PM |
| Day | Dose | dose | dose | dose | dose | dose | dose | dose | dose |
| 1 | 58 | 29 | 29 | 15 | 14 | 15 | 14 | 9.67 | 9.67 |
| 2 | 56 | 28 | 28 | 14 | 14 | 14 | 14 | 9.33 | 9.33 |
| 3 | 54 | 27 | 27 | 14 | 13 | 14 | 13 | 9 | 9 |
| 4 | 52 | 26 | 26 | 13 | 13 | 13 | 13 | 8.67 | 8.67 |
| 5 | 50 | 25 | 25 | 13 | 12 | 13 | 12 | 8.33 | 8.33 |
| 6 | 48 | 24 | 24 | 12 | 12 | 12 | 12 | 8 | 8 |
| 7 | 46 | 23 | 23 | 12 | 11 | 12 | 11 | 7.67 | 7.67 |
| 8 | 44 | 22 | 22 | 11 | 11 | 11 | 11 | 7.33 | 7.33 |
| 9 | 42 | 21 | 21 | 11 | 10 | 11 | 10 | 7 | 7 |
| 10 | 40 | 20 | 20 | 10 | 10 | 10 | 10 | 6.67 | 6.67 |
| 11 | 38 | 19 | 19 | 10 | 9 | 10 | 9 | 6.33 | 6.33 |
| 12 | 36 | 18 | 18 | 9 | 9 | 9 | 9 | 6 | 6 |
| 13 | 34 | 17 | 17 | 9 | 8 | 9 | 8 | 5.67 | 5.67 |
| 14 | 32 | 16 | 16 | 8 | 8 | 8 | 8 | 5.33 | 5.33 |
| 15 | 30 | 15 | 15 | 8 | 7 | 8 | 7 | 5 | 5 |
| 16 | 28 | 14 | 14 | 7 | 7 | 7 | 7 | 4.67 | 4.67 |
| 17 | 26 | 13 | 13 | 7 | 6 | 7 | 6 | 4.33 | 4.33 |
| 18 | 24 | 12 | 12 | 6 | 6 | 6 | 6 | 4 | 4 |
| 19 | 22 | 11 | 11 | 6 | 5 | 6 | 5 | 3.67 | 3.67 |
| 20 | 20 | 10 | 10 | 5 | 5 | 5 | 5 | 3.33 | 3.33 |
| 21 | 18 | 9 | 9 | 5 | 4 | 5 | 4 | 3 | 3 |
| 22 | 16 | 8 | 8 | 4 | 4 | 4 | 4 | 2.67 | 2.67 |
| 23 | 14 | 7 | 7 | 4 | 3 | 4 | 3 | 2.33 | 2.33 |
| 24 | 12 | 6 | 6 | 3 | 3 | 3 | 3 | 2 | 2 |
| 25 | 10 | 5 | 5 | 3 | 2 | 3 | 2 | 1.67 | 1.67 |
| 26 | 8 | 4 | 4 | 2 | 2 | 2 | 2 | 1.33 | 1.33 |
| 27 | 6 | 3 | 3 | 2 | 1 | 2 | 1 | 1 | 1 |
| 28 | 4 | 2 | 2 | 1 | 1 | 1 | 1 | 0.67 | 0.67 |
| 29 | 2 | 1 | 1 | 1 | 0 | 1 | 0 | 0.33 | 0.33 |

TABLE 3

| | q12 hour | | | | | | | |
|---|---|---|---|---|---|---|---|---|
| | morphine (e.g. | | q12 hour | | q12 hour | | q12 hour | |
| | MS Contin) | | hydrocodone | | oxycodone | | levorphanol | |
| | | PM | AM | PM | AM | PM | AM | PM |
| Day | M dose | dose | dose | dose | dose | dose | dose | dose |
| 1 | 29 | 29 | 10 | 9 | 10 | 9 | 4 | 3.5 |
| 2 | 28 | 28 | 9 | 9 | 9 | 9 | 3.5 | 3.5 |
| 3 | 27 | 27 | 9 | 8 | 9 | 8 | 3.5 | 3 |
| 4 | 26 | 26 | 8 | 8 | 8 | 8 | 3 | 3 |
| 5 | 25 | 25 | 8 | 7 | 8 | 7 | 3 | 2.5 |
| 6 | 24 | 24 | 7 | 7 | 7 | 7 | 2.5 | 2.5 |
| 7 | 23 | 23 | 7 | 6 | 7 | 6 | 2.5 | 2 |
| 8 | 22 | 22 | 6 | 6 | 6 | 6 | 2 | 2 |
| 9 | 21 | 21 | 6 | 5 | 6 | 5 | 2 | 1.5 |
| 10 | 20 | 20 | 5 | 5 | 5 | 5 | 1.5 | 1.5 |
| 11 | 19 | 19 | 5 | 4 | 5 | 4 | 1.5 | 1 |
| 12 | 18 | 18 | 4 | 4 | 4 | 4 | 1 | 1 |
| 13 | 17 | 17 | 4 | 3 | 4 | 3 | 1 | .5 |
| 14 | 16 | 16 | 3 | 3 | 3 | 3 | .5 | .5 |
| 15 | 15 | 15 | 3 | 2 | 3 | 2 | .5 | 0 |
| 16 | 14 | 14 | 2 | 2 | 2 | 2 | | |
| 17 | 13 | 13 | 2 | 1 | 2 | 1 | | |
| 18 | 12 | 12 | 1 | 1 | 1 | 1 | | |
| 19 | 11 | 11 | 1 | 0 | 1 | 0 | | |
| 20 | 10 | 10 | | | | | | |
| 21 | 9 | 9 | | | | | | |
| 22 | 8 | 8 | | | | | | |
| 23 | 7 | 7 | | | | | | |
| 24 | 6 | 6 | | | | | | |
| 25 | 5 | 5 | | | | | | |
| 26 | 4 | 4 | | | | | | |
| 27 | 3 | 3 | | | | | | |
| 28 | 2 | 2 | | | | | | |

As an example of this invention, the medication taper pack containing a 24-hour morphine formulation for a patient using 60 mg per day (as shown in Table 2) would contain 29 different daily doses in a sequence that reduces each daily dose by 1 mg. Other examples of other opioid treatments are shown in Tables 2 and 3. Although it would be preferred that the total daily dose decrease be constant across the taper period as shown in the Tables 2 and 3, it would also be acceptable to have less frequent than daily dose changes as long as each total daily dose decrease is less than a therapeutic opioid dose (i.e., it is a sub-therapeutic change in dose). For example, in the case of morphine, the generally accepted oral therapeutic dose for the treatment of moderate to severe pain is 15 mg. Therefore, a sub-therapeutic dose would generally be considered equivalent to less than 15 mg oral morphine.

While a steady rate total daily dose decrease of less than 5 mg oral morphine equivalents per day is acceptable, for example, it is preferred to decrease the total daily dose of an opioid by an average of 2 morphine equivalents or less per day as long as a single total daily dose decrease less than 15 morphine equivalents per day occurs during the taper period. As examples, a 5 mg oral morphine dose decrease every 5 days would be acceptable, as would a 3 mg oral morphine dose decrease every 3 days.

Thus, this method is not limited to a specific opioid subtype since all mu agonist opioids like those identified above have extremely similar clinical effects and are generally considered interchangeable if an appropriate equianalgesic dose conversion is performed.

If a subject is taking a total daily dose of less than 60 mg of morphine equivalents, then the tapering schedule should be adjusted to begin at a dose that is 1-2 mg of morphine equivalents less than the current total daily dose (e.g., start sub-therapeutic steady-rate total daily dose opioid taper at 38-39 mg morphine if subject is taking 40 mg morphine at baseline).

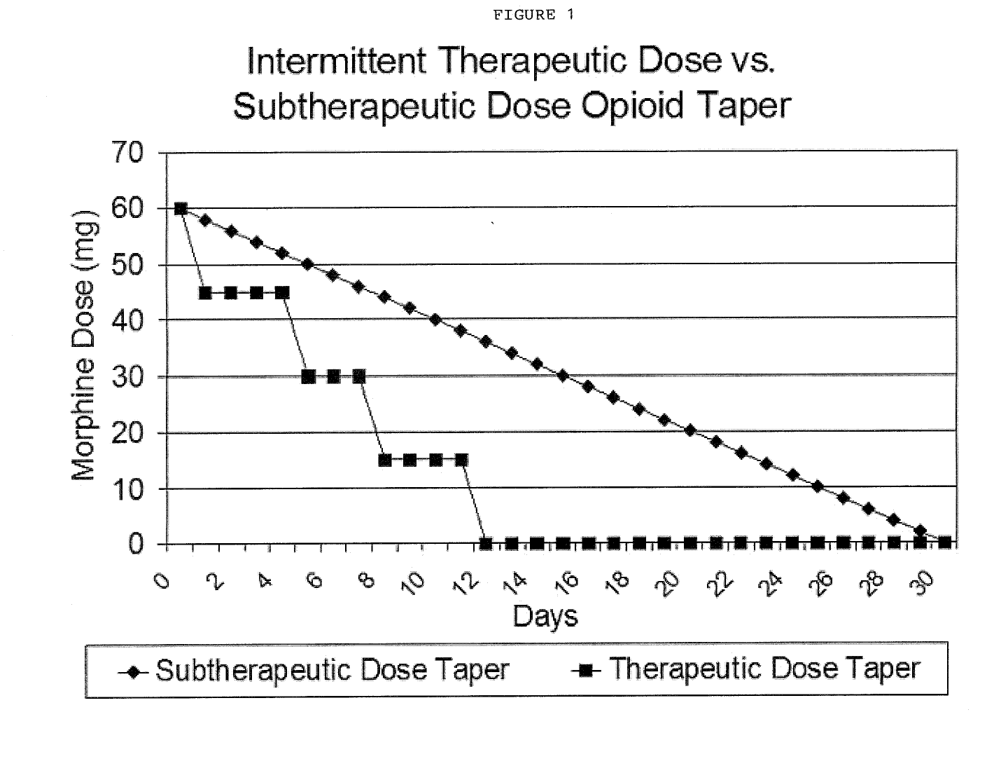

A graphical summary of the present steady state, sub-therapeutic total daily dose tapering method for 60 mg immediate release morphine, in comparison to the intermittent, therapeutic dose step tapering method recommended by the United States Veterans Affairs Administration, Clinical Practice Guideline for the Management of Opioid Therapy for Chronic Pain, 2003; Version 1.0, page 51-52. Available at http://www.healthquality.va.gov/cot/cot fulltest.pdf. Accessed Nov. 29, 2009, is shown in FIG. 1.

As explained herein, it an essential aspect of the present invention that the total daily dose tapering method include the use of sub-therapeutic amounts of pharmacological agent. A therapeutic dose amount is known to medical professionals who prescribe these types of drugs. As explained earlier herein in connection with the foregoing examples, the therapeutic oral dose amount of morphine (immediate release formulation) is 15 mg. Accordingly, any sub-therapeutic amount is a dose amount less than 15 mg morphine (immediate release formulation). A sub-therapeutic amount may be about half of the therapeutic morphine amount (i.e., about 7.5 mg). Still further alternatively, the sub-therapeutic amount should be no more than about 50%, or about 33%, or about 25% of a therapeutic amount or less generally.

Also as described earlier, the specific examples set forth above relate to opioids. Other tolerance-inducing pharmacological agents include benzodiazepines. Therapeutic does amounts of these tolerance-inducing pharmacological agents can be calculated in the same way as shown above for opioids. The tolerance-inducing doses are all known to those in the medical area who prescribe these sorts of pharmacological agents. These therapeutic dose amounts are also well documented in the literature and examples are set forth below in Table 4.

### Benzodiazepines

TABLE 4

| | | Minimum Amounts | |
|---|---|---|---|
| | | of the | |
| | | Benzodiazepine | |
| | | Required to | |
| | | Accomplish a | |
| Drug Name | Therapeutic Indications | Therapeutic Purpose | |
| Alprazolam | anxiolytic | 0.25 | mg |
| Chlordiazepoxide | anxiolytic | 5 | mg |
| Clonazepam | anxiolytic, anticonvulsant | 0.5 | mg |
| Clorazepate | anxiolytic, anticonvulsant | 7.5 | mg |
| Diazepam | anxiolytic, anticonvulsant, | 2 | mg |
| | muscle relaxant | | |
| Estazolam | hypnotic | 0.5 | mg |
| Flurazepam | hypnotic | 15 | mg |
| Lorazepam | anxiolytic, anticonvulsant | 0.5 | mg |
| Oxazepam | anxiolytic | 10 | mg |
| Temazepam | hypnotic, anticonvulsant | 7.5 | mg |
| Triazolam | hypnotic | 0.125 | mg |
| Atypical | | | |
| Benzodiazepines | | | |
| Eszopiclone | hypnotic | 1 | mg |
| Zaleplon | hypnotic | 5 | mg |
| Zolpidem | hypnotic | 5 | mg |

Diazepam was the first marketed member of the benzodiazepine class of drugs and has been studied extensively in the treatment of a variety of medical conditions. The medical conditions where diazepam has been shown to be effective include as an anxiolytic, anticonvulsant and muscle relaxant. For these three chronic conditions, the therapeutic dose of diazepam can be defined as 2 mg. Using the formulas above, the maximal daily taper regimen would be a 33.3% decrease (i.e. 0.67 mg per day) over a period of 3 days.

While the invention has been described with reference to specific embodiments thereof, it will be understood that numerous variations, modifications and additional embodiments are possible, and accordingly, all such variations, modifications, and embodiments are to be regarded as being within the spirit and scope of the invention.

What is claimed is:

1. A method for decreasing the use of tolerance-inducing pharmacological agent comprising the steps of:
identifying a tolerance-inducing pharmacological agent;
identifying a therapeutic dose amount of the tolerance-inducing pharmacological agent, the therapeutic dose amount being the minimum amount of the tolerance-inducing pharmacological agent required to accomplish a therapeutic purpose;
creating a multiple total daily dose regimen of the tolerance-inducing pharmacological agent that comprises a plurality of successively smaller total daily dose amounts of the tolerance-inducing pharmacological agent, with the total daily dose amounts decreasing at a substantially steady rate;
further wherein the total daily dose amount decreases are in decrements of sub-therapeutic amounts of the tolerance-inducing pharmacological agent.

2. The method described in claim 1 wherein, the tolerance-inducing pharmacological agent is selected from the group consisting of opioids and benzodiazepines.

3. The method described in claim 1, wherein the tolerance-inducing pharmacological agent is an opioid.

4. The method described in claim 1, wherein the total daily dose regimen comprises more than a therapeutic dose amount of the tolerance-inducing pharmacological agent.

5. The method described in claim 1, wherein the successively smaller decrements of the total daily dose regimen of the tolerance-inducing pharmacological agent comprises a decreasing dose amount for each day of the regimen.

6. The method described in claim 5, wherein the total daily dose regimen of the tolerance-inducing pharmacological agent comprises multiple dose amounts per day, and the sum of the dose amounts for each day is less than the sum of the total daily dose amounts of the previous day.

7. The method described in claim 1, wherein the total daily dose of the tolerance-inducing pharmacological agent is reduced to zero at the end of the regimen.

8. The method described in claim 3, wherein the therapeutic dose amount is at least one therapeutic dose, and each successively smaller sub-therapeutic dose amount decrement is less than or equal to about 33.3% of the therapeutic dose.

9. The method described in claim 3, wherein the total daily dose is provided as an oral formulation selected from the group consisting of a pill, capsule, tablet, lozenge, aerosol or trans-oral formulation.

10. The method described in claim 3, wherein the opioid is selected from the group consisting of morphine, fentanyl, hydrocodone, hydromorphine, levorphanol, meperidine, methadone, oxycodone, tapentadol and tramadol.

11. The method described in claim 1, wherein the therapeutic total daily dose taper regimen lasts from 3-7 days per therapeutic dose.

12. The method described in claim 1, wherein the therapeutic total daily dose taper regimen lasts more than 7 days per therapeutic dose.

13. The method described in claim 1, wherein the successively smaller total daily dose decreases are substantially uniform.

14. The method described in claim 1, wherein each successively smaller sub-therapeutic dose amount is about half of a therapeutic dose amount or less.

15. The method described in claim 16, wherein each successively smaller sub-therapeutic amount is about 25% of a therapeutic dose amount or less.
